(19) United States
(12) Patent Application Publication
(10) Pub. No.: US 2023/0338591 A1
(43) Pub. Date: Oct. 26, 2023

(54) Sanitizing Misting Device

(71) Applicant: Sulestine Scott, Ripley, TN (US)

(72) Inventor: Sulestine Scott, Ripley, TN (US)

(21) Appl. No.: 18/171,489

(22) Filed: Feb. 20, 2023

Related U.S. Application Data
(60) Provisional application No. 63/333,075, filed on Apr. 20, 2022.

(51) Int. Cl. A61L 2/00; A61L 2/10; A61L 2/26

(52) U.S. Cl. CPC A61L 2/0088; A61L 2/10; A61L 2/26; A61L 2202/14

(57) ABSTRACT

The present invention relates to a device and method for automatically sanitizing individuals when entering a home or other building. The device comprises a relatively small and cylindrical housing configured to fit within the top threshold of a door frame. Alternatively, the device can be hung from a ceiling. The device features a motion sensor for detecting when a person approaches and walks through the door, and a control panel for immediately activating a gentle mist of disinfectant directed at the person. The device includes an air-directed blower for directing warm air and effectively killing germs, viruses and/or bacteria. The device uses a disinfectant tank for storing disinfectant solution and a mist pump/electronic motor for actuating a mist of disinfectant under the top threshold of the door frame.

### CROSS-REFERENCE TO RELATED APPLICATION

The present application claims priority to, and the benefit of, U.S. Provisional Application No. 63/333,075, which was filed on Apr. 18, 2022 and is incorporated herein by reference in its entirety.

### FIELD OF THE INVENTION

The present invention relates generally to the field of disinfecting systems. More specifically, the present invention relates to a sanitizing, misting device that is designed to be attached to a top threshold of a regular door frame. The sanitizing, misting device features a motion sensor for detecting a motion near the door frame, such as when an individual walks through the door and automatically activates the misting device for spraying and airing a directed gentle mist of disinfectant at the individual. A control panel, coupled with the motion sensor, activates the gentle mist. The sprayed disinfectant mist can disinfect substantially the entire body of the individual and can rid him or her from any germs, bacteria and viruses. The sanitizing misting device of the present invention offers an easy way for individuals to maintain a disinfected and sanitary environment. Accordingly, the present disclosure makes specific reference thereto. Nonetheless, it is to be appreciated that aspects of the present invention are also equally applicable to other like applications, devices, and methods of manufacture.

### BACKGROUND

By way of background, various kinds of infectious diseases transmit from one person to another due to spread of harmful pathogens such as germs, bacteria, viruses and more. Also, in the times of pandemics, such as an epidemic of an infectious disease such as COVID-19 or other viruses, it can be nearly impossible for an individual to protect himself or herself from infected individuals, and to prohibit transmission of infections. It can be extremely difficult to keep the hands and body disinfected at all times in order to prevent the spread of infections, germs, viruses, bacteria, etc.

Various products such as hand sanitizers, sanitizing sprays and more are available in the market. These products can be used by individuals to keep their hands disinfected or to sanitize a desired surface area/object. However, hand sanitizers are generally used to keep hands disinfected, but not the complete body of an individual. Also, the sanitizing sprays are manually used to spray disinfectant over a surface area or an object in order to ensure a disinfected surface/object. However, individuals may not always remember to carry hand sanitizers and sanitizer bottles with them while travelling to places, such as going to someone's home or the like.

Individuals that come to visit a home may not wash their hands enough and risk spreading harmful airborne pathogens. Not only can the hands be a source of transmission of harmful pathogens, but the complete body of an individual can carry viruses, bacteria and more. Entering and exiting a home or other building constantly can lead to diseases being brought in to other places. This increases the spread of infectious diseases and makes it difficult to control the infections. Also, with ease of transmission, it can be extremely difficult to control pandemics, thereby impacting the lives of millions of individuals.

Therefore, there exists a long felt need in the art for a device that helps to sanitize the complete body of an individual, and ensures no harmful pathogens such as germs, bacteria and more are present or retained on the individual's body. There is also a long felt need in the art for a sanitization device that does not require users to carry hand sanitizers, sanitizing spray and the like with them. Additionally, there is a long felt need in the art for a sanitizing device that makes it easy to keep the hands and body disinfected. Moreover, there is a long felt need in the art for a sanitizing device that can be used for sanitizing individuals entering or exiting a home or a building, thereby preventing spread of harmful pathogens. Further, there is a long felt need in the art for a sanitizing device that helps in controlling the transmission of infectious diseases. Furthermore, there is a long felt need in the art for a sanitizing device that helps in controlling pandemics. Finally, there is a long felt need in the art for a sanitizing device that is easily installable and offers and easy way for individuals to maintain a disinfected and sanitary environment.

The subject matter disclosed and claimed herein, in one embodiment thereof, comprises a sanitizing device for spraying a mist of a disinfecting solution. The sanitizing device further comprising a housing that includes a plurality of mist spray nozzles disposed at the bottom of the housing for spraying a disinfecting mist, a motion sensor within the housing for detecting motion within a predefined range, wherein the sanitizing device automatically sprays the disinfecting mist in a downward direction upon detecting motion, and a control panel disposed within the housing for activating and deactivating spraying of the disinfecting mist based on detected motion, wherein the activation is for a predetermined time interval or duration.

In this manner, the novel sanitizing misting device of the present invention accomplishes all of the forgoing objectives, and provides a relatively safe, easy and convenient solution to ensure the hands, and substantially the entire body, of an individual remain disinfected and free of any germs, viruses, bacteria or other harmful pathogens. The sanitizing misting device of the present invention is also user-friendly, as it does not require users to carry hand sanitizers or other similar products while travelling to another person's home. Additionally, the sanitizing device automatically sprays the disinfectant over an individual upon entering and/or exiting a home or building, and does not require any manual effort from users to disinfectant visitors. The sanitizing misting device of the present invention helps in the containment and mitigation of pandemics and other infectious diseases.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a sanitizing device for spraying a mist of a disinfecting solution toward and on an individual. The sanitizing device is designed to be installed within a top threshold of a door frame. The sanitizing device, further comprising a housing that includes a plurality of mist spray nozzles disposed at the bottom of the housing for spraying disinfecting mist, a motion sensor within the housing for detecting motion near the door frame within a predefined range, wherein the sanitizing device automatically sprays the disinfecting mist in a downward direction upon detecting motion, and a control panel disposed within the housing for activating and deactivating spraying of the disinfecting mist based on detected motion wherein the activation is for a predetermined time duration or interval. The sanitizing device can be connected to a disinfecting solution tank for supply of the disinfecting solution, and the sanitizing device can include a pair of door grips that are selectively-attached to the door frame to mount the sanitizing device to the door frame.

In yet another embodiment, the sanitizing device also includes a plurality of air-directed blower holes disposed at the bottom of the housing to direct warm air to dry out or evaporate any moisture caused due to the fine mist. The air is produced by a built-in air-directing blower within the housing.

In yet another embodiment, the sanitizing device also includes a UV disinfectant emitter for emitting UV disinfectant rays. The UV disinfectant emitter is not automatically activated by the motion sensor, but can be activated manually (i.e. actuated) using a control.

In yet another embodiment of the present invention, a door mist device is disclosed. The door mist is a sanitizing, misting device with an air directed blower. The device can be designed in a cylindrical shape and can be made to fit in the top threshold of a regular door frame. The device includes a built-in motion sensor that based upon detecting motion near the door, activates a gentle mist of a disinfectant towards a person standing at the door. The mist droplets can be fine in size and thus do not wet the clothes of the person. The mist droplets are formed by an electric motor or mist pump connected to the disinfectant supply tank.

In yet another embodiment, the mist droplets can be directed air for a predetermined time interval or duration, such as five seconds or eight seconds. In one potential embodiment, the time ranges between five to ten seconds. The control panel of the device can be configured with the predetermined time value.

In yet another embodiment of the present invention, a method for making an entrance through a sanitizing entrance is described. The method includes installing a sanitizing device at the top of the door frame such that the mist nozzles are directed in a downward direction, detecting a motion near the door frame on which the device is installed, automatically or autonomously activating a gentle mist and directed air in the downward direction for a predetermined time, enabling the person standing at the door frame to be sanitized and automatically deactivating the gentle mist directed air after the predetermined time is over. In yet another embodiment, the method includes directing warm air for drying out or evaporating any moisture on the clothes of a person due to the gentle mist.

In yet another embodiment of the present invention, a sanitizing misting device configured to be hung from a ceiling using hanging rods is disclosed. The device is designed to direct air and a gentle mist in a downward direction to disinfect a person standing therebelow. The sanitizing device includes a motion sensor for detecting motion near the device, and a plurality of mist spray holes for automatically directing air and a gentle mist upon detecting motion by the motion sensor.

In a further embodiment of the present invention, the device includes a built-in disinfectant solution tank and a mist sprayer for spraying a gentle mist of disinfectant.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a sanitizing misting device of the present invention in accordance with the disclosed architecture;

FIG. 2 illustrates a perspective view of another potential embodiment of a sanitizing misting device of the present invention in accordance with the disclosed architecture;

FIG. 3 illustrates a perspective view showing one potential embodiment of the sanitizing misting device installed within the top threshold of a doorframe and spraying mist in accordance with the disclosed architecture;

FIG. 4 illustrates a perspective view showing the hanging sanitizing misting device installed on a ceiling in accordance with the disclosed architecture; and

FIG. 5 illustrates a flow diagram showing determination of timing of the directed air and gentle mist based on detected motion by the motion sensor of the sanitizing misting device of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a device that helps in sanitization of the complete body of an individual and ensures no harmful pathogens such as germs, bacteria and more are present or retained on the individual's body. There is also a long felt need in the art for a sanitization device that does not require users to carry hand sanitizers, sanitizing spray and the like with them. Additionally, there is a long felt need in the art for a sanitizing device that makes it easy to keep the hands and body disinfected. Moreover, there is a long felt need in the art for a sanitizing device that can be used for sanitizing individuals entering or exiting a home or a building, thereby preventing the spread of harmful pathogens. Further, there is a long felt need in the art for a sanitizing device that helps in controlling the transmission of infectious diseases. Furthermore, there is a long felt need in the art for a sanitizing device that helps in controlling pandemics. Finally, there is a long felt need in the art for a sanitizing device that is easily installable and offers an easy way for individuals to maintain a disinfected and sanitary environment.

The present invention, in one exemplary embodiment, is a novel door mist device. The door mist is a sanitizing, misting device with an air directed blower. The device is designed in a cylindrical shape and is made to fit in, or on, the top threshold of a regular door frame. The device includes a built-in motion sensor that, based on detecting motion near the door, activates a gentle mist of a disinfectant and directs same toward a person standing at the door. The mist droplets are fine in size and do not wet the clothes of the person. The mist droplets are formed by a mist pump connected to the disinfectant supply tank.

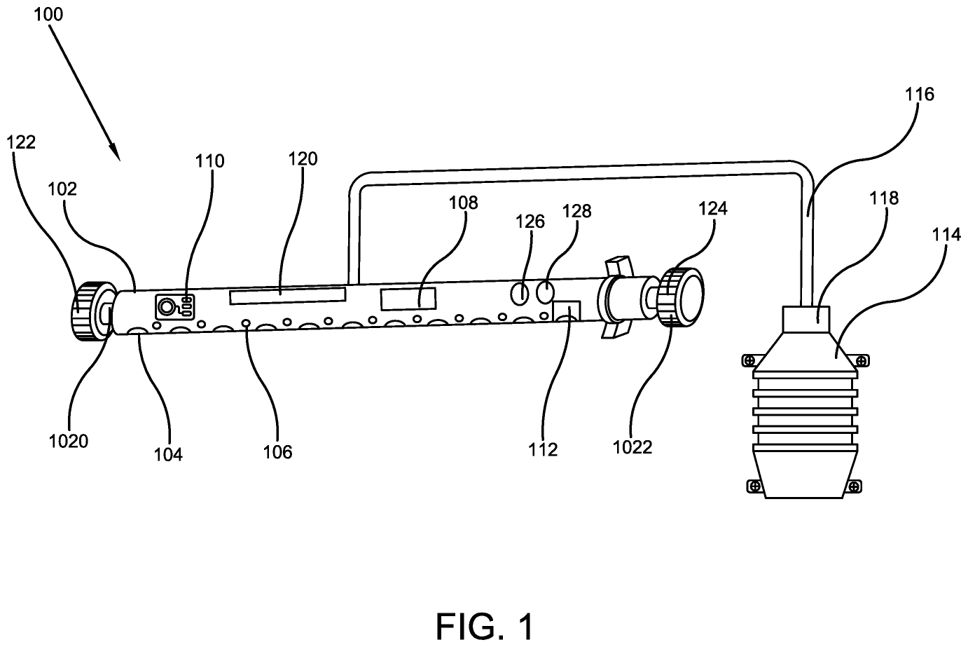

Referring initially to the drawings, FIG. 1 illustrates a perspective view of one potential embodiment of the sanitizing misting device of the present invention in accordance with the disclosed architecture. The sanitizing misting device 100 of the present embodiment is designed to spray and direct air and a gentle mist of disinfectant at a person entering through a door on which the sanitizing device 100 is installed. The device 100 is designed to be installed on the top of a door frame in order to spray the disinfectant material in the form of a gentle mist at a person passing through the door. More specifically, the device 100 includes a housing 102 in which the components of the misting device 100 reside. The housing 102 provides a cover to safeguard the mist sprayer holes 104, air directing openings 106, motion sensor 108, and control panel 110. The sanitizing misting device 100 includes an optional ultraviolet (UV) disinfectant emitter 112 to provide an additional virus-killing device that is particularly suited for inanimate objects. The UV disinfectant emitter 112 can be controlled manually by an operator, so that the UV disinfectant emitter is prohibited from emitting when a human passes through the door or the entrance. It is to be appreciated that ultraviolet germicidal irradiation is a disinfection method that uses short-wavelength ultraviolet light to kill or inactivate microorganisms by destroying nucleic acids and disrupting their DNA, leaving them unable to perform vital cellular functions.

A plurality of mist sprayer nozzles 104 are disposed at the bottom of the housing 102, and can be used for spraying a gentle mist of disinfectant towards a person. The disinfectant spraying device 100 is connected to a disinfectant tank 114 through a hose 116 that supplies the disinfectant mist to the plurality of spray holes 104 for spraying in a downward direction. The disinfectant tank 114 can have a volume capacity generally from about five liters to about fifteen liters. The tank 114 can be placed on the ground, or alternatively can be in the form of a built-in tank within the housing 102, and can be refilled by an inlet hose. The direction of the spray holes 104 are designated in a downward direction, thereby enabling a downward spray of mist of disinfectant material. An electric motor/mist pump 118 can be used with the disinfectant tank 114 for producing a mist of the disinfectant stored in the disinfectant tank 114.

The mist can be sprayed at angles up to ten degrees on both sides (i.e. fore and aft) of the longitudinal vertical axis of the spraying device 100, thereby enabling the spraying mist to disinfect the entire body (i.e. substantially the entire exterior) of a person passing under the spraying device 100 for killing germs, viruses and bacteria. In order to quickly absorb or evaporate the moisture of mist sprayed on the body of the person, a plurality of air directed openings 106 disposed on the device 100 can direct warm air toward the body of the person, thereby enabling the clothes of the person to dry quickly. A built-in air directing blower 120 can be used for producing the warm air for directing the air through the air directed openings 106. It should be noted that the air-directed or blower openings 106 can be absent in one of the variants, where only the mist nozzles 104 are disposed at the bottom of the housing 102.

One feature of the disinfectant mist spray device 100 includes the built-in motion sensor 108. The motion sensor 108 detects a motion in the range of one to four feet from the door threshold and sends an instruction signal to the control panel 110. The control panel 110, upon receiving the instruction signal, activates the mist spray of disinfectant material from the plurality of mist spray holes 104. It should be noted that the control panel 110 can be configured with a predetermined spray time or duration, such as between three and eight seconds, for example, to spray disinfectant mist and then can direct warm air for the same duration or a different predetermined time period. Manual operation is not required to operate the spraying of disinfectant mist and for directed blowing of air. Using the motion sensor 108, the device 100 enables an automatic operation for efficient and easy disinfectant of the individuals passing through a threshold of a door.

In the present embodiment, the disinfectant spray device 100 includes a pair of door frame grips 122, 124, thereby enabling the device 100 to be installed within the top threshold of a door frame. As shown, the first door frame grip 122 at the first end 1020 and the second door frame grip 124 at the second end 1022 can be mounted to the door frame using mechanical fasteners to mount the device 100 to the door frame.

A pair of LED indicators 126, 128 can be disposed on the housing 102, indicating the operation and status of the spraying device 100. A first LED 126 can flash in a first color, such as green color, indicating running status of the device 100, and indicating that the mist is being sprayed by the device 100, and a second LED 128 can flash in a second color, such as red indicating that there is a fault in the device 100 and the disinfectant mist cannot be sprayed.

For operating the optional UV emitter 112, an operator can selectively actuate a manual or remote control for emitting UV disinfectant light on luggage and/or other material or inanimate items for additional disinfecting and safety. It should be appreciated that both the disinfectant mist spray and warm air can be directed to a passing individual, or selectively can be deactivated based on the requirements of users. The device 100 provides a touchless disinfectant spray that does not require any manual effort, and ensures that each person entering an enclosed area under the device 100 is properly sanitized and disinfected. The sanitizing misting device 100 effectively solves the problem of disinfecting and sterilization of the entrance and exit of a key protection area, and effectively blocks the spread of viruses and bacteria. When the device 100 is installed above a door frame, the device 100 effectively forms a disinfecting and sterilization pass-through for ingress and egress.

The housing 102 can come in different colors and sizes to fit door frames of different sizes. Also, the housing 102 is lightweight and can be made from a lightweight yet sturdy material such as a metal, heavy duty plastic or the like. The pressure of the mist can be controlled using the electric motor 118.

The motion sensor 108 can be an infrared motion sensor that includes a thin pyroelectric film material and detects infrared rays emitting from individuals approaching the device 100. Alternatively, the motion sensor 108 can be a microwave sensor that works on the principle of Doppler radar.

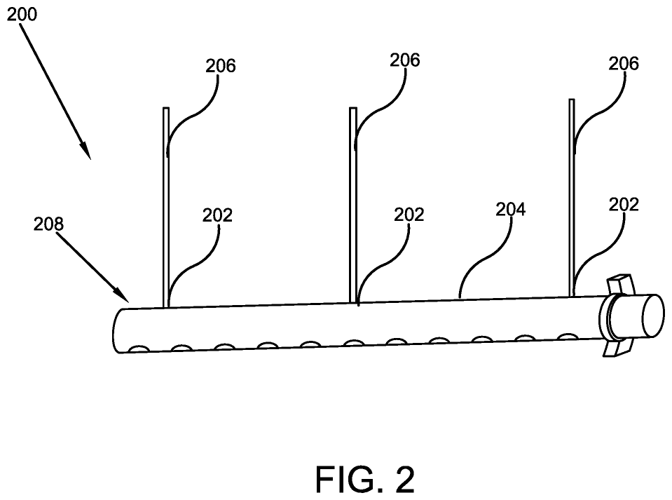

FIG. 2 illustrates a perspective view of another potential embodiment of the sanitizing misting device of the present invention in accordance with the disclosed architecture. In the present embodiment, the sanitizing misting device 200 is designed to hang from the ceiling rather than being mounted on the top of a door frame. The sanitizing misting device 200 includes a plurality of sockets 202 on the top surface 204 of the housing 208, thereby enabling the hanging arms 206 to hang the device 200 from the ceiling. This is useful in areas where door frames do not have enough height or space to accommodate the sanitizing device. It should be appreciated that the components such as the mist sprayer holes, air directed blowers, motion sensor, and control panel work in the same manner as the other embodiments described supra and are not repeated for the sake of clarity. Further, in this present embodiment, the electric motor/mist pipe and the tank or reservoir are also housed within the housing 208 of the device 200.

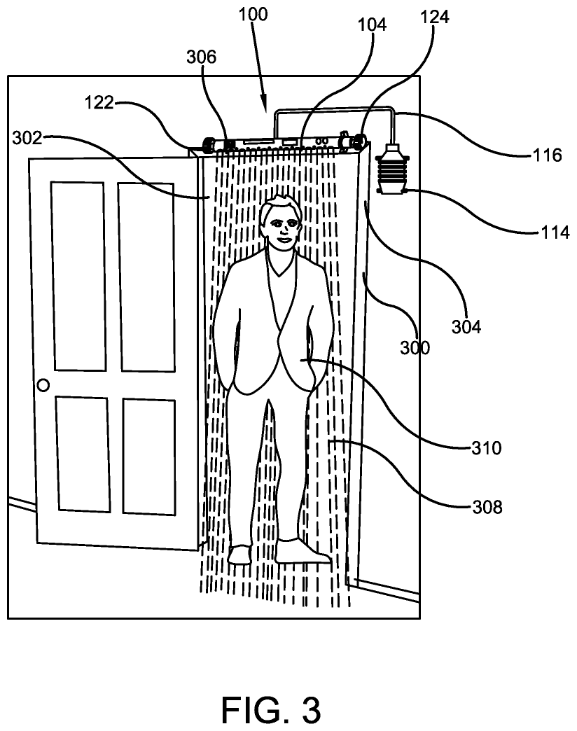

FIG. 3 illustrates a perspective view showing one potential embodiment of the sanitizing misting device 100 installed within the top threshold of a door frame and spraying mist in accordance with the disclosed architecture. As shown, the first door frame grip 122 is mounted to the first side 302 of the door frame 300, and the second door frame grip 124 is mounted to the second side 304 of the door frame 300, for installing the sanitizing misting device 100 within the top threshold 306 of the door frame 300. The device 100 is installed near the top threshold 306 so that the downward sprayed mist 308 completely or substantially disinfects a person 310 standing below the sanitizing device 100. The disinfectant tank 114 can be connected to the device 100 through the hose 116 for supplying the disinfectant mist 308 for spraying at the door 300. It should be appreciated that the built-in motion sensor 108, as shown in FIG. 1, detects the motion of the person 310 on either side of the door frame 300 up to four feet for starting to spray the mist 308 from spray nozzles 104 for disinfecting the person 310 standing at the door 300. Also, as mentioned the spray is performed for a predetermined time, such as five seconds. It should be appreciated that the any disinfectant material, such as one or a combination of Hydrogen Peroxide, Quaternary Ammonium Compounds (Quats), Chlorine Compounds or Phenolic Compounds can be used with the device 100 of the present invention. Further, using the electric motor/mist pump 118, a volume of the spray dispensed within the predetermined time interval can be adjusted.

The disinfecting spray device 100 designed to be installed at doors and can be readily installed at the entrances and exits of public buildings, hospitals, pharmacies, airports, supermarkets, airports, transit stations, etc. The disinfecting spray device 100 is designed to require little to no maintenance, and the installation offers individuals maximum protection against viruses and diseases during their daily movements.

The size of the mist droplets 308 sprayed by the spray nozzles 104 is extremely fine, and can range in the size of 5-20 microns, thus the spray does not wet the clothes. The minimal moisture that remains on the clothes is dried by the warm air directly blown by the air-directed openings 106 as shown in FIG. 1.

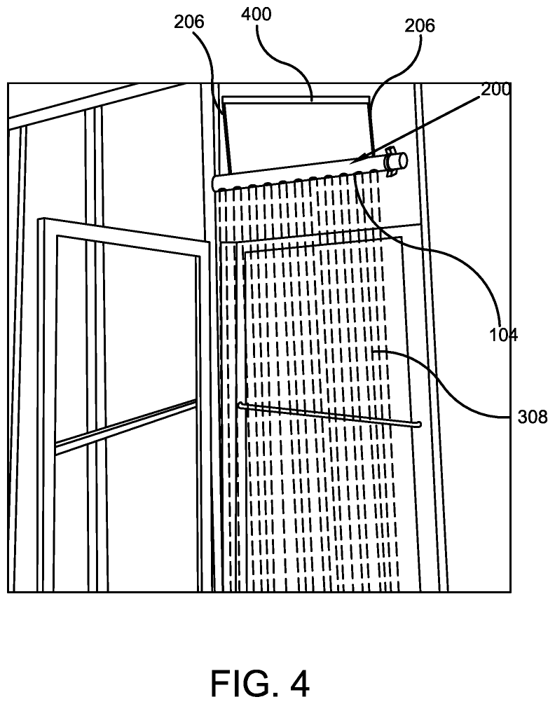

FIG. 4 illustrates a perspective view showing the hanging sanitizing misting device 200 installed on ceiling 400 in accordance with the disclosed architecture. In the present embodiment, the sanitizing misting device 200 is installed on the ceiling 400 and is hung using the hanging arms 206. The spray nozzles 104 spray the gentle mist 308 in a downward direction for automatically sanitizing individuals standing below the device 200. It should be appreciated that the height of the device 200 can be adjusted based on the ceiling height for effective sanitization of individuals. Further, the device 200 is useful inside a premises where a door may not be necessarily present, and where there is a requirement for automatic sanitization and disinfecting of individuals.

In one embodiment, the sanitizing devices 100, 200 include a sensor to monitor the solution levels in the disinfecting tank (whether the tank is built-in or is an external tank) and a trouble-shooting sensor to detect any mechanical errors in the devices 100, 200. Notifications for each of the solution level, troubleshooting or the like can also be transmitted through a visible or audible alarm, or can be transmitted to a smartphone through a wireless communication channel made through a wireless communication module present within the device 100, 200.

Further, the devices 100, 200 can be of any size and shape that can fit and be installed on the door frames and ceilings used in homes, retail shops, malls, hospitals etc. The devices 100, 200 reduce the spread of germs, viruses and bacteria by sanitizing individuals entering and exiting.

The mist spray devices 100, 200 include a sufficient number of nozzles to thoroughly sanitize the individuals standing below the devices 100, 200 in a minimum amount of time and without any manual intervention. The mist spray includes antimicrobial properties, thereby enabling the effective sanitization of the person.

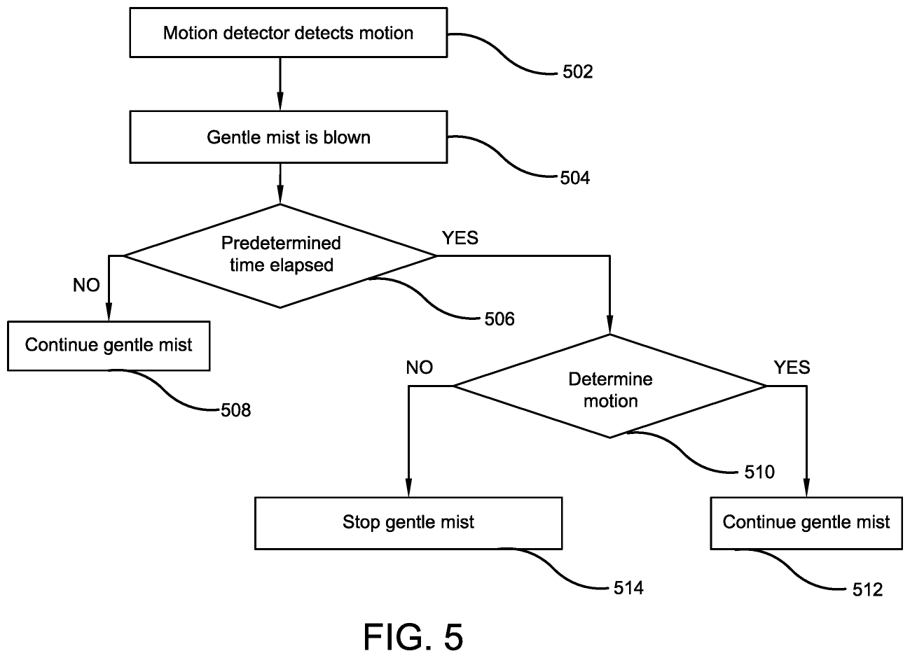

FIG. 5 illustrates a flow diagram showing a determination of timing of the directed air and gentle mist based on detected motion by the motion sensor of the sanitizing misting device of the present invention in accordance with the disclosed architecture. As shown, initially the motion detector detects motion near the device and instructs the control panel of the detected motion (Block 502). Then, the control panel activates the spray nozzles, the directed air and the gentle mist (Block 504). Thereafter, the control panel determines if the predetermined time of gentle mist has elapsed or not (Block 506). If it is determined that the time interval has not elapsed, then the gentle mist continues to be directed and blown by the sanitizing device (Block 508). If it is determined that the predetermined time interval has elapsed, then additional determinations are assessed if motion is detected by the motion detector (Block 510). If further motion is detected, then the gentle mist is continue to be sprayed, thereby enabling additional individuals passing below the device to be sanitized (Block 512). Otherwise, the gentle mist is terminated and may be followed by directed warm air (Block 514).

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “sanitizing misting device”, “sanitizing device”, “misting device”, “disinfectant spraying device”, “spraying device”, “disinfectant mist spray device”, “disinfecting spray device” and “device”, are interchangeable and refer to the sanitizing misting device 100, 200 of the present invention.

Notwithstanding the forgoing, the sanitizing misting device 100, 200 of the present invention can be of any suitable size and configuration as is known in the art without affecting the overall concept of the invention, provided that it accomplishes the above-stated objectives. One of ordinary skill in the art will appreciate that the size, configuration, and material of the sanitizing misting device 100, 200 as shown in the FIGS. are for illustrative purposes only, and that many other sizes and shapes of the sanitizing misting device 100, 200 are well within the scope of the present disclosure. Although the dimensions of the sanitizing misting device 100, 200 are important design parameters for user convenience, the sanitizing misting device 100, 200 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What includes been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sanitizing misting device comprising:
a housing including a plurality of mist sprayer nozzles, a motion sensor, and a control panel, wherein said plurality of mist sprayer nozzles disposed at a bottom of said housing and directed downward for spraying a mist of disinfectant below said housing;
a disinfectant tank including a hose connected to said housing for supplying disinfectant from said tank to said housing;
an electric motor and mist pump dispenses said mist of disinfectant; and
a motion sensor for sensing movement of a person proximal to said housing, whereupon said control panel actuates said dispensing.

2. The sanitizing misting device of claim 1, wherein said dispensing is for a predetermined time interval.

3. The sanitizing misting device of claim 2, wherein said time interval is adjustable.

4. The sanitizing misting device of claim 1, wherein said mist of disinfectant covers substantially the entire exterior of a person passing under said housing.

5. The sanitizing misting device of claim 1 further comprising a blower and a plurality of directed openings for directing warm air toward the person passing under said housing.

6. The sanitizing misting device of claim 1, wherein said disinfectant tank includes a volume capacity generally from about five liters to about fifteen liters.

7. The sanitizing misting device of claim 1, wherein said disinfectant tank built-in said housing.

8. The sanitizing misting device of claim 1, wherein said mist of disinfectant sprayed at angles up to ten degrees on both sides fore and aft of the longitudinal vertical axis of said housing.

9. The sanitizing misting device of claim 1 further comprising an ultraviolet (UV) disinfectant emitter to provide an additional virus killing mechanism.

10. The sanitizing misting device of claim 9, wherein said UV disinfectant emitter is manually actuated.

11. A sanitizing misting device comprising:
a plurality of mist sprayer nozzles, wherein said plurality of mist sprayer nozzles disposed at a bottom of said sanitizing misting device and directed downward for spraying a mist of disinfectant below said sanitizing misting device;
a motion sensor;
a control panel;
an electric motor;
a mist pump; and
a pair of door frame grips for mounting the sanitizing misting device to a door threshold.

12. The sanitizing misting device of claim 11, wherein said dispensing is for a predetermined time interval.

13. The sanitizing misting device of claim 12, wherein said mist of disinfectant covers substantially the entire exterior of a person passing under said misting device.

14. The sanitizing misting device of claim 11 further comprising a disinfectant tank including a hose connected to said misting device for supplying disinfectant from said tank to said misting device.

15. The sanitizing misting device of claim 11 further comprising a blower and a plurality of directed openings for directing warm air toward the person passing under said misting device.

16. The sanitizing misting device of claim 13, wherein said mist of disinfectant sprayed at angles up to ten degrees on both sides fore and aft of the longitudinal vertical axis of said misting device.

17. The sanitizing misting device of claim 11 further comprising an ultraviolet (UV) disinfectant emitter to provide an additional virus killing mechanism.

18. The sanitizing misting device of claim 17, wherein said UV disinfectant emitter is manually actuated.

19. A method for creating a sanitizing entrance threshold, the method comprising the steps of:
installing a sanitizing device at an upper edge of a door frame threshold;
directing a plurality of mist nozzles in a downward direction;
detecting a motion of a person near said door frame threshold;
activating automatically a sanitizing mist and directed air in said downward direction for a predetermined time interval; and
covering a person passing though said door frame threshold with said sanitizing mist.

20. The method of claim 19 further comprising a step of directing warm air from said sanitizing device for evaporating moisture from said sanitizing mist.
